(19) United States
(12) Patent Application Publication
(10) Pub. No.: US 2005/0281882 A1
(43) Pub. Date: Dec. 22, 2005

(54) Matrix made of a polysaccharide modified under an electron beam with a functional organosilicon compound

(75) Inventors: Ian Harrison, Poissy (FR); Jean-Francois Sassi, Saint-Roman En Jarez (FR); Christian Priou, Charbonnieres-Les-Bains (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: RHODIA CHIMIE, BOULOGNE BILLANCOURT CEDEX (FR)

(21) Appl. No.: 11/066,194

(22) Filed: Feb. 25, 2005

Related U.S. Application Data
(60) Provisional application No. 60/548,546, filed on Feb. 27, 2004.

(51) Int. Cl. C08B 11/00; C08B 11/20; C07G 17/00; C08B 37/00; A61K 9/14

(52) U.S. Cl. 424/488; 536/85; 536/43; 536/114

(57) ABSTRACT

Water-soluble or water-dispersible matrix, made of a polysaccharide modified under an electron beam with an organosilicon compound chosen from organosilanes and/or polyorganosiloxanes having at least one functional group capable of reacting and/or interacting with said polysaccharide. Use of the matrix as stabilizing agent in the preparation of simple emulsions, in particular of the water-in-oil type, or multiple emulsions, in particular of the water-in-oil-in-water type.

The subject of the present invention is a matrix made of a polysaccharide modified under an electron beam with a functional organosilicon compound chosen from functional organosilanes and/or functional polyorganosiloxanes; this matrix may be used as a stabilizing agent in the preparation of a simple, in particular inverse emulsion, or of a multiple emulsion, in particular of the water-in-oil-in-water type.

It is known to depolymerize polysaccharides under electron beams (WO 04/000885), and thus to obtain polysaccharides of lower molecular mass.

It is known to graft an ethylenic monomer onto a polysaccharide under an electron beam, with depolymerization of the polysaccharide (WO 04/001386).

It is also known to crosslink, under an electron beam, polyorganosiloxanes comprising crosslinkable epoxy or vinyl-functional groups in the presence of an initiator based on a boron derivative that is activable under an electron beam (EP 1114067 B1).

The aim of the invention is to produce a polysaccharide matrix with organosilicon groups chosen from lipophilic organosilanes and lipophilic polyorganosiloxanes, the matrix exhibiting good solubility or dispersibility in water or aqueous media.

A first subject of the invention consists of a matrix (M), that is water-soluble or water-dispersible, made of at least one polysaccharide (PSA) modified under an electron beam with at least one organosilicon compound having at least one functional group and/or at least one functional group capable of reacting and/or interacting with said polysaccharide (PSA), which organosilicon compound is chosen from organosilanes (S) and/or polyorganosiloxanes (POS) having at least one functional group and/or at least one functional group capable of reacting and/or interacting with said polysaccharide (PSA).

The expression “water-soluble or water-dispersible” means here that said matrix (M) made of modified polysaccharide is not capable of forming a two-phase macroscopic solution at 25° C. when it is added to water or to an aqueous solution.

In the text which follows, the expression “lipophilic” will be used here as antonym of the term “hydrophilic”, that is to say has no affinity for water; this means that the compound or the group considered is capable of forming, at a concentration of 10% by weight, a two-phase macroscopic solution in distilled water at 25° C.

Said polysaccharide (PSA) which can be used to obtain the matrix (M) according to the invention is a homopolysaccharide or a heteropolysaccharide; it may be linear or branched, nonionic or ionic; it may be optionally substituted and/or modified with nonionic or (potentially) ionic groups other than lipophilic polyorganosiloxane groups.

Preferably, said polysaccharide (PSA), or its backbone, comprises similar or different glycosyl units joined by β(1-4) bonds. It may additionally comprise, apart from the β(1-4) bonds, other bonds, in particular β(1-3) and/or β(1-6).

The weight-average molecular mass of the polysaccharide (PSA) may range from 1000 to 5 000 000, preferably from 1000 to 3 000 000 g/mol measured for example by size exclusion chromatography (SEC) with MALS (Multiple Angle Laser Scattering) detection.

Said similar or different glycosyl units may be in particular hexose and/or pentose units.

Among the hexose units (similar or different), there may be mentioned in particular D-glucose, D- or L-galactose, D-mannose, D- or L-fucose and L-rhamnose units and the like.

Among the pentose units (similar or different), there may be mentioned in particular D-xylose and L- or D-arabinose units and the like.

Hydroxyl functional groups of the glycosyl units may be modified and/or substituted with nonionic, ionic or potentially ionic groups. However, preferably, the average number of hydroxyl functional groups of the glycosyl units substituted or modified with said nonionic, ionic or potentially ionic groups is less than 3, preferably less than 2, and most particularly less than 1.5.

In the case of nonionic modifying groups, these may in particular be linked to the carbon atoms of the sugar backbone either directly or via —O— bonds.

Among the nonionic groups, there may be mentioned:
alkyl groups comprising from 1 to 22 carbon atoms, optionally interrupted by one or more heteroatoms of oxygen and/or nitrogen,
aryl or arylalkyl groups comprising from 6 to 12 carbon atoms
hydroxyalkyl or cyanoalkyl groups comprising from 1 to 6 carbon atoms
“ester” groups obtained by replacing the hydrogen of a hydroxyl functional group —OH of the polysaccharide backbone with a group comprising at least acid motif containing in particular carbon, sulfur or phosphorus, such as in particular carbonyl R—(CO)—, sulfonyl R—SO₂—, phosphoryl R₂P(O)— and hydroxyphosphoryl R—P(O)(OH)— groups, and acid groups forming “ester” motifs with the remaining oxygen atoms of the polysaccharide backbone. The R, alkyl, alkenyl or aryl group may comprise from 1 to 20 carbon atoms; it may additionally comprise a heteroatom, of nitrogen for example, directly linked to a carbonyl or sulfonyl motif, and the like, and thus form bonds of the urethane type, and the like.

By way of example, there may be mentioned:
methyl, ethyl, propyl, isopropyl, butyl, hexyl, octyl, dodecyl, octadecyl and phenyl groups, linked to a carbon atom of the polysaccharide backbone via an ether, ester, amide or urethane bond
cyanoethyl, hydroxyethyl, hydroxypropyl and hydroxybutyl groups, linked to a carbon atom of the polysaccharide backbone via an —O— bond
“ester” groups chosen from acetate, propanoate, trifluoroacetate, 2-(2-hydroxy-1-oxopropoxy)propanoate, acetate phthalate, lactate, glycolate, pyruvate, crotonate, isovalerate, cinnamate, formate, salicylate, carbamate, methylcarbamate, benzoate, gluconate, methanesulfonate and toluenesulfonate groups; the hemiester groups of fumaric, malonic, itaconic, oxalic, maleic, succinic, tartaric, aspartic, glutamic and malic acids; there may be mentioned more particularly the substituent groups acetate, hemiacetate and 2-(2-hydroxy-1-oxopropoxy)-propanoate.

The degree of modification MS of a polysaccharide with a nonionic modifying group corresponds to the average number of moles of precursor of the nonionic modifying group which has reacted per glycosyl unit.

The degree of modification MS can vary according to the nature of the precursor of said modifying group.

If said precursor is not capable of forming new reactive hydroxyl groups (precursor of alkylation for example), the degree of modification with the nonionic groups is less than 3 by definition.

If said precursor is capable of forming new reactive hydroxyl groups (precursor of hydroxyalkylation for example), the degree of modification MS is theoretically not limited; it may for example be up to 6, preferably up to 2. This level is generally at least 0.001, preferably at least 0.01.

Among the anionic or potentially ionic groups, there may be mentioned those containing one or more carboxylate, sulfonate, sulfate, phosphate and phosphonate functional groups, and the like.

There may be mentioned in particular those of formula
—[—CH₂—CH(R)—O]ₓ—(CH₂)_{y}—COOH or
—[—CH₂—CH(R)—O]ₓ—(CH₂)_{y}—COOM, where
R is a hydrogen atom or an alkyl radical containing from 1 to 4 carbon atoms
x is an integer ranging from 0 to 5
y is an integer ranging from 0 to 5
M represents an alkali metal

There may be mentioned most particularly the carboxyl groups —COO⁻Na⁺ linked directly to a carbon atom of the sugar backbone, carboxymethyl groups (sodium salt) —CH₂—COO⁻Na⁺ linked to a carbon atom of the sugar backbone via an —O— bond.

Among the cationic or potentially cationic groups, there may be mentioned those containing one or more amino, ammonium, phosphonium or pyridinium functional groups, and the like.

There may be mentioned in particular the cationic or potentially cationic groups of formula
—NH₂
—[—CH₂—CH(R)—O]ₓ—(CH₂)_{y}—COA-R′—N(R″)₂
—[—CH₂—CH(R)—O]ₓ—(CH₂)_{y}—COA-R′—N⁺(R′″)₃X⁻
—[—CH₂—CH(R)—O]ₓ—(CH₂)_{y}—COA-R′—NH—R″″—N(R″)₂
—[—CH₂—CH(R)—O]ₓ—R′—N(R″)₂
—[—CH₂—CH(R)—O]ₓ—R′—N⁺(R′″)₃X⁻
—[—CH₂—CH(R)—Oₓ—R′—NH—R″″—N(R″)₂
—[—CH₂—CH(R)—O]ₓ—Y—R″
where
R is a hydrogen atom or an alkyl radical containing from 1 to 4 carbon atoms
x is an integer ranging from 0 to 5
y is an integer ranging from 0 to 5
R′ is an alkylene radical containing from 1 to 12 carbon atoms, optionally bearing one or more substituents OH
the radicals R″, which are similar or different, represent a hydrogen atom, an alkyl radical containing from 1 to 18 carbon atoms
the radicals R′″, which are similar or different, represent an alkyl radical containing from 1 to 18 carbon atoms
R″″ is a linear, branched or cyclic alkylene radical containing from 1 to 6 carbon atoms
A represents O or NH
Y is a heterocyclic aliphatic group comprising from 5 to 20 carbon atoms and a nitrogen heteroatom
X⁻ is a counterion, preferably halide (chloride, bromide, iodide in particular),

and N-alkylpyridinium-yl groups in which the alkyl radical contains from 1 to 18 carbon atoms, with a counterion, preferably halide (chloride, bromide, iodide in particular).

Among the cationic or potentially cationic groups, there may be mentioned most particularly:
those of formula
   —NH₂
   —CH₂—CONH—(CH₂)₂—N(CH₃)₂
   —CH₂—COO—(CH₂)₂—NH(CH₂)₂—N(CH₃)₂
   —CH₂—CONH—(CH₂)₃—NH(CH₂)₂—N(CH₃)₂
   —CH₂—CONH—(CH₂)₂—NH(CH₂)₂—N(CH₃)₂
   —CH₂—CONH—(CH₂)₂—N⁺(CH₃)₃Cl⁻
   —CH₂—CONH—(CH₂)₃—N⁺(CH₃)₃Cl⁻
   —(CH₂)₂—N(CH₃)₂
   —(CH₂)₂—NH(CH₂)₂—N(CH₃)₂
   —(CH₂)₂—N⁺(CH₃)₃ Cl⁻
most particularly 2-hydroxypropyltrimethylammonium chloride
   —CH₂—CH(OH)—CH₂—N⁺(CH₃)₃Cl⁻
the pyridinium-yl groups such as N-methyl-pyridinium-yl, of formula 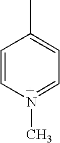
with a chloride counterion
hindered amino groups such as those derived from amines HALS, of general formula: 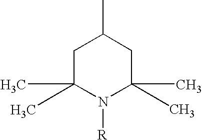
where R represents CH3 or H.

Among the betaine groups, there may be mentioned most particularly the functional groups of formula:

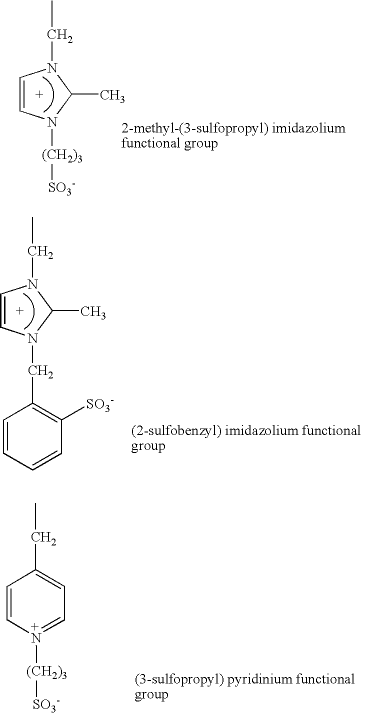

—(CH₂)₂—N⁺(CH₃)₂—(CH₂)₂—COO— ethyldimethylammonium betaine functional group

—(CH₂)₂—N⁺(CH₃)₂—(CH₂)₃—SO₃— sulfopropyldimethylammonium functional group

The degree of substitution DS corresponds to the average number of hydroxyl functional groups of the glycosyl units substituted with said ionic or ionizable group(s), per glycosyl unit.

It is less than 3, preferably less than 2.

Among the polysaccharides (PSA) which may be used to obtain the matrix (M) of the invention, there may be mentioned natural or synthetic polysaccharides, optionally modified and/or substituted by the chemical route with nonionic or (potentially) ionic groups other than lipophilic polyorganosiloxane groups, and/or degraded (depolymerized) by acid or base hydrolysis, or by an oxidative, thermal or enzymatic route, or under an electron beam.

By way of examples, there may be mentioned:
polysaccharides whose backbone contains only β(1-4) bonds, such as celluloses optionally modified or substituted with one or more nonionic groups (in particular acetate; hydroxyalkyl, preferably hydroxyethyl, hydroxypropyl; hydroxypolyethoxy), (potentially) anionic groups (in particular carboxyalkyl, preferably carboxymethyl), (potentially) cationic groups (2-hydroxypropyltrimethylammonium chloride in particular):

There may be mentioned in particular:
cellulose monoacetates, having a degree of substitution of 0.3 to less than 1.2, preferably of 0.3 to 1.
hydroxypropylated celluloses having a degree of modification of the order of 0.2 to 1.5, such as Primaflo HP22 marketed by Aqualon
hydroxyethylcellulose such as Cellosize HEC QP 100M-H marketed by Dow
carboxymethylcelluloses having a degree of substitution of 0.05 to 1.2, preferably of 0.05 to 1, such as Blanose Cellulose gum from Hercules and Liberty 3794 from Aqualon
2-hydroxypropyltrimethylammonium chloride celluloses, such as AMERCHOL JR-400 marketed by Amerchol.
Galactomannans (in particular guar gum), optionally modified or substituted with one or more nonionic groups (preferably hydroxyalkyl, in particular hydroxypropyl), (potentially) anionic groups (preferably carboxyalkyl, in particular carboxymethyl), cationic groups (preferably cationic hydroxyalkylgalactomannan, in particular hydroxypropyltrimethylammonium chloride), and/or optionally depolymerized groups.

There may be mentioned in particular:
guar gums, in particular as a powder, such as JAGUAR 6003VT marketed by Rhodia, as split grains, such as ECOPOL 3650 marketed by Economy Polymers, GALACTOSOL 252 marketed by Aqualon, SUPERCOL GUAR GUM marketed by Aqualon
modified guars, such as carboxyalkyl guars (carboxymethyl guars, carboxypropyl guars), carboxymethylhydroxypropyl guars, hydroxyalkyl guars (hydroxyethyl guars, hydroxypropyl guars, hydroxybutyl guars), hydroxypropyltrimethylammonium chloride guars, most preferably hydroxypropyl guars having a degree of substitution of less than 0.6, such as JAGUAR 8000 marketed by Rhodia
guars depolymerized by the oxidative route (having a few COOH⁺ functional groups resulting from depolymerization in an oxidizing medium), such as MEYPRO-GAT 7, MEYPRO-GAT 20, MEYPRO-GAT 30 marketed by Rhodia
hydroxypropylated depolymerized guars having a degree of modification of the order of 0.01 to 0.8
carboxymethylated depolymerized guars having a degree of substitution of the order of 0.05 to 1.6 such as MEYPRO-GUM R 600 marketed by Rhodia
cationized depolymerized guars having a degree of substitution of the order of 0.04 to 0.17, preferably 0.06 and 0.14, such as MEYPRO-COAT 21 marketed by Rhodia, AquaCat CG 518 marketed by Aqualon

Dextrins optionally containing hydroxyethyl or hydroxypropyl groups or quaternized aminoalkyl groups (degradation of starches, optionally chemically modified with hydroxyethyl or hydroxypropyl groups or quaternized aminoalkyl groups).

Xyloglycans such as the tamarind gum Instasol 1200 from Saiguru Food, MEYPRO-GUM T12 marketed by Rhodia.

According to the invention, the polysaccharide (PSA) is modified under an electron beam with a functional organosilicon compound chosen from functional organosilanes (S) and functional polyorganosiloxanes (POS).

Preferably, the functional group(s) of said organosilicon compound are capable of reacting and/or interacting with the polysaccharide (PSA) according to an ionic or free-radical mechanism.

Most preferably, this may be epoxyfunctional groups (capable of reacting and/or interacting according to an ionic mechanism), a vinyl functional group or alkenyl functional groups (capable of reacting and/or interacting according to a free-radical mechanism). Most preferably, it is an epoxyfunctional group.

The functional organosilane (S) which may be used may contain from 1 to 3 functional groups capable of reacting or interacting with the polysaccharide (PSA).

Said functional organosilane (S) may be represented by the formula
R¹R′¹R″¹SiY′
the symbol R¹ representing:
   a linear or branched alkyl or alkoxy radical containing 1 to 8 carbon atoms, optionally substituted with at least one halogen, preferably fluorine, the alkyl radicals being preferably methyl, ethyl, propyl, octyl, 3,3,3-trifluoropropyl, methoxy, ethoxy, isopropoxy,
   an optionally substituted cycloalkyl radical containing between 5 and 8 cyclic carbon atoms,
   an aryl radical containing between 6 and 12 carbon atoms which may be substituted, preferably phenyl, tolyl or dichlorophenyl,
   an aralkyl part having an alkyl part containing between 5 and 14 carbon atoms and an aryl part containing between 6 and 12 carbon atoms, which is optionally substituted on the aryl part with halogens, alkyls and/or alkoxyls containing 1 to 3 carbon atoms,
the symbols R′¹ and R″¹, which are similar or different, representing R¹ or Y′
the symbol Y′ representing a functional group capable of reacting and/or interacting with the polysaccharide (PSA).

Preferably, the symbols R′¹ and R″¹, which are similar or different, represent the symbol R¹.

Most preferably, the silane (S) has the formula (CH₃)₃SiY′.

The functional polyorganosiloxane (POS) is preferably at least partially linear or cyclic.

It may contain on average from 2 to 1000 siloxy motifs, preferably from 3 to 100 siloxy motifs per macromolecular chain.

It has, at the chain end(s) and/or in its chain, at least one functional group capable of reacting and/or interacting with the polysaccharide (PSA).

Advantageously, said functional polyorganosiloxane (POS) has on average from 1 to 10, preferably from 1 to 3, more particularly 1 or 2 functional groups capable of reacting and/or interacting with the polysaccharide (PSA).

Advantageously, said functional polyorganosiloxane (POS) comprises motifs of formula (IV) and/or is terminated by motifs of formula (V) below: 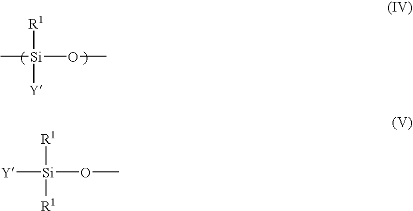
in which formulae:
the symbols R¹ are similar or different and represent:
   a linear or branched alkyl or alkoxy radical containing 1 to 8 carbon atoms, optionally substituted with at least one halogen, preferably. fluorine, the alkyl radicals being preferably methyl, ethyl, propyl, octyl, 3,3,3-trifluoropropyl, methoxy, ethoxy, isopropoxy,
   a cycloalkyl radical containing between 5 and 8 cyclic carbon atoms, which is optionally substituted,
   an aryl radical containing between 6 and 12 carbon atoms, which may be substituted, preferably phenyl, tolyl or dichlorophenyl,
   an aralkyl part having an alkyl part containing between 5 and 14 carbon atoms and an aryl part containing between 6 and 12 carbon atoms, which is optionally substituted on the aryl part with halogens, alkyls and/or alkoxyls containing 1 to 3 carbon atoms,
the symbols Y′ are similar or different and represent
   a radical R¹
   a functional group capable of reacting and/or interacting with the polysaccharide (PSA), at least one of the symbols Y′ being different from R¹.

Advantageously, said polyorganosiloxane (POS) contains from 1 to 10, preferably from 1 to 3, most particularly 1 or 2 radicals Y′ different from R¹.

The linear polyorganosiloxanes may be oils having a dynamic viscosity at 25° C. of the order of 10 to 10 000 mPa.s at 25° C., generally of the order of 50 to 1000 mPa.s at 25° C.

In the case of cyclic polyorganosiloxanes, these consist of motifs (IV) which may be, for example, of the dialkylsiloxy or alkylarylsiloxy type. These cyclic polyorganosiloxanes have a viscosity of the order of 1 to 5000 mPa.s.

The dynamic viscosity at 25° C. of said polyorganosiloxane (POS) can be measured with the aid of a BROOKFIELD viscometer, according to the AFNOR NFT 76 102 standard of February 1972.

Preferably, the functional groups (Y′) of the organosilane (S) or of the polyorganosiloxane (POS) react and/or interact with the polysaccharide (PSA) according to an ionic or free-radical mechanism.

Most preferably, this may be epoxyfunctional groups (capable of reacting and/or interacting according to an ionic mechanism), a vinyl functional group or alkenyl functional groups (capable of reacting and/or interacting according to a free-radical mechanism). Most preferably, it involves an epoxyfunctional group.

Preferably, the symbols Y′, and more generally the functional groups of the organosilanes (S) or of the polyorganosiloxanes (POS) are similar or different and represent:
the vinyl radical —CH═CH₂,
and/or an epoxy and/or alkenyl and/or alkenyloxy and/or alkenylcarbonyloxy and/or alkenylcarbonylamino radical linked to the silicon atom of the organosilane or to a silicon atom of the polyorganosiloxane via a divalent radical containing from 2 to 20 carbon atoms and which may contain at least one heteroatom, preferably oxygen.

Most particularly, this may involve epoxyfunctional groups.

Among the symbols Y′ or epoxyfunctional groups, there may be mentioned the groups of the following formulae:

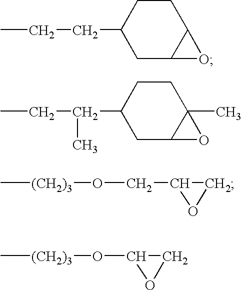

Among the symbols Y′ or alkenyl functional groups, there may be mentioned the groups of the following formulae:
—(CH₂)₃—O—CH═CH₂ —(CH₂)₃—O—R²—O—CH═CH₂
—(CH₂)₃—O—CH═CH—R —(CH₂)₃—(OR′²)ₙ—O—CH═CH₂
—(CH₂)₃—O—(O)C—CH═CH₂ —(CH₂)₃—O—(O)C—C(R)═CH₂
—(CH₂)₃—NH—(O)C—C(R)═CH₂ —(CH₂)₃—NH—(O)C—C(R)═CH₂

in which:
R² represents:
   a linear or branched C₁-C₁₂ alkylene radical, which is optionally substituted
   or a C₅-C₁₂ arylene radical, preferably phenylene, which is optionally substituted, preferably with one to three C₁-C₆ alkyl groups,
R′² represents a linear or branched C₂-C₃ alkyl radical, with n ranging from 2 to 100,
R represents a linear or branched C₁-C₆ alkyl radical, preferably methyl.

For good implementation of the invention, the matrix (M) is obtained by irradiation, under an electron beam, of a layer, of uniform thickness, of a homogeneous mixture of the polysaccharide (PSA) and of the organosilicon compound having at least one functional group capable of reacting and/or interacting with said polysaccharide (PSA),
the polysaccharide (PSA)/organosilicon compound mass ratio ranging from 1/99 to 99/1, preferably from 70/30 to 90/10
the uniform thickness of the mixture layer ranging up to 3 cm, preferably up to 1.5 cm, more particularly ranging from 100 μm to 1.5 cm
the radiation dose absorbed ranging from 1 to less than 100 kilogray (kGy), preferably from 1 to 50 kGy.

When said organosilicon compound is an organosilane (S), the polysaccharide (PSA)/organosilane (S) mass ratio is favorably from 50/50 to 99/1, preferably from 70/30 to 90/10.

The homogeneous mixture of polysaccharide (PSA) and organosilicon compound subjected to the irradiation operation may be in solid or liquid form; most preferably it is in solid form.

Said homogeneous mixture subjected to irradiation may be obtained beforehand by mixing the two constituents, with suitable stirring, in the presence or otherwise of solvent.

In the absence of solvent, the mixture may be prepared by adding the organosilicon compound to the polysaccharide (PSA) (as a powder, as granules, as flakes and the like), and homogenizing, with stirring, with the aid of a device of the Lödige Brabender type, and the like.

A homogeneous mixture may also be obtained by dissolving the polysaccharide (PSA) in a solvent such as acetone, dimethylacetamide (DMAC), dimethylformamide (DMF) or a mixture of solvents, in particular isopropanol/water mixtures (for example in an isopropanol/water mass ratio ranging from 10/90 to 90/10), and adding the silicon compound, with stirring, with the aid of a device of the turbine or anchor type, and the like.

After bringing into contact, with stirring, (for example for 15 minutes to 3 hours at a temperature of 15° C. to 100° C., most generally at room temperature), the solvent or the mixture of solvents may be evaporated, if desired. Advantageously, the homogeneous mixture obtained (preferably as a powder) is then uniformly deposited, in a thickness which may range up to 3 cm, preferably up to 1.5 cm, more particularly ranging from 100 μm to 1.5 cm, on the plateau of a conveyor belt, and then conveyed under the irradiation window of an electron bombardment apparatus. This operation may be carried out in the presence or in the absence of air.

Various types of apparatus for irradiation by electron bombardment may be used. They may be high-energy apparatus, such as the RHODOTRON ELECTRON BEAM ACCELERATOR provided by IBA. It is also possible to use a low-energy apparatus, such as EZCure provided by ESI (Energy Sciences. Inc.).

The speed of conveying the homogeneous mixture and the intensity of the current are set according to the apparatus in order to obtain the desired dose of radiation absorbed.

The operation for irradiating the mixture itself lasts for less than one second, generally from 0.001 to 0.5 second.

The above implementation conditions used make it possible to control the modification of the polysaccharide (PSA) with the organosilicon compound, this being in order to avoid or to limit crosslinking reactions which result in the formation of insoluble species or species which are soluble with difficulty in water or aqueous media.

It is assumed that the matrix obtained is formed of a mixture of several polysaccharide or organosilicon macromolecular species, in particular of macromolecules of polysaccharide functionalized with one or more lipophilic organosilane and/or polyorganosiloxane groups, and/or of macromolecules of polysaccharides which are optionally partially depolymerized and/or of functionalized polyorganosiloxanes which are optionally partially depolymerized.

According to a variant embodiment, the irradiation operation is carried out in the presence of an activator capable of being activated by an electron beam.

This may be in particular a boron derivative of formula M⁺B(Ar)₄⁻ where:
M⁺, an entity bearing a positive charge, is chosen from an alkali metal of groups IA and IIA of the Periodic Table (CAS version),
Ar is an aromatic derivative, optionally substituted with at least one substituent chosen from a fluorine radical, a chlorine radical, a linear or branched alkyl chain, which may itself be substituted with at least one electron-attracting group such as CₙF₂ₙ₊₁ with n being between 1 and 18 (for example: CF₃, C₃F₇, C₂F₅, C₈F₁₇) F, and OCF₃.

M may be chosen in particular from lithium, sodium, cesium or potassium.

By way of example, the boron derivative of the initiator according to the invention is of formula: LiB(C₆F₅)₄, KB(C₆F₅)₄, KB(C₆H₃(CF₃)₂) ₄ and CsB(C₆F₅)₄.

The initiator concentration may range from 0.01 to 5%, preferably from 0.01 to 1% by mass relative to the mass of polysaccharide and of silicon compound used.

The initiator is preferably used in solution in a solvent. The weight ratios between the initiator, on the one hand, and the solvent, on the other hand, are between 0.1 and 99 parts per 100 parts of solvent and preferably from 10 to 50 parts.

The solvents may be in particular alcohols, esters, ethers and ketones.

The alcohols commonly used are para-tolylethanol, isopropylbenzyl alcohol, benzyl alcohol, methanol, ethanol, propanol, isopropanol and butanol. The ethers commonly used are 2-methoxyethanol, 2-ethoxyethanol, diethylene glycol. The customary esters are dibutyl maleate, dimethylethyl malonate, methyl salicylate, dioctyl adipate, butyl tartrate, ethyl lactate, n-butyl lactate and isopropyl lactate. There may also be mentioned acetonitrile, benzonitrile, acetone, cyclohexanone and tetrahydrofuran.

If desired, the solvent for the initiator may then be optionally removed by evaporation.

According to a second subject of the invention, the matrix (M) made of polysaccharide modified under an electron beam may be used as stabilizing agent in the preparation of simple or multiple emulsions. This may be a simple, invert emulsion, in particular of the water-in-oil type, or a multiple emulsion, for example a double emulsion, in particular of the water-in-oil-in-water type.

The matrix (M) according to the invention is in particular advantageous for stabilizing invert emulsions (Ei) of the water-in-oil type.

The quantity of matrix (M) which may be used for stabilizing invert emulsions (Ei) of an aqueous phase (Wi) in a hydrophobic phase (O) corresponds to a ratio of the mass of stabilizing matrix (M) to the mass of hydrophobic phase (O) which may range from 0.1/100 to 500/100, preferably from 0.5/100 to 100/100, most particularly from 0.5/100 to 50/100.

The mass ratio of the aqueous phase (Wi) to the hydrophobic phase (O) may range from 5/95 to 95/5, preferably from 30/70 to 80/20.

The mean size of the aqueous droplets (Wi) of the invert emulsion (Ei) may range up to 10 μm, preferably from 0.05 μm to 5 μm, and more preferably from 0.1 to 1 μm.

The mean size corresponds to the median diameter by volume (d50), which represents the diameter of the particle equal to 50% of the cumulative distribution; it may be measured for example with a Horiba particle size analyzer or an optical microscope.

The aqueous phase (Wi) has a pH which may range from 0 to 14, preferably from 2 to 11, more preferably from 5 to 11.

It may contain additives which make it possible to adjust the osmotic pressure, such as salts (sodium chloride or sulfate, calcium chloride and the like) or sugars (glucose) or polysaccharides (dextran and the like).

It may also contain buffering agents, hydrophilic active substances, in particular antibacterial agents such as methylchloroisothiazolinone and methylisothiazolinone (KATHON® CG marketed by Rohm and Haas), other water-soluble or water-dispersible materials, and hydrophobic substances which are insoluble in the hydrophobic phase (O).

The hydrophobic phase (O) may be any material or mixture of liquid materials (or in a liquid form) or meltable materials which are insoluble in the aqueous phase (Wi).

The material constituting the hydrophobic phase (O) may be considered as being insoluble when less than 15%, preferably less than 10%, of its weight is soluble in the aqueous phase (Wi).

Said hydrophobic phase (O) preferably has a melting point of less than or equal to 100° C., more particularly of less than or equal to 80° C.

It may be for example:
an oil, a wax or a resin made of a nonionic, ionic or ionogenic, linear, cyclic, branched or crosslinked polyorganosiloxane, in particular nonionic or aminated, preferably linear polyorganosiloxanes
an oil or an organic wax such as
   mono-, di- or triglycerides of C₁-C₃₀ carboxylic acids or mixtures thereof, such as vegetable oils
   technical oils, such as boiled linseed oils, blown linseed oils or linseed standoil marketed by NOVANCE
   sucroesters, sucroglycerides
   C₁-C₃₀ alcohol esters of C₁-C₃₀ carboxylic or C₂-C₃₀ dicarboxylic acids
   ethylene or propylene glycol monoesters or diesters of C₁-C₃₀ carboxylic acids
   propylene glycols of C₄-C₂₀ alkyl ethers
   C₈-C₃₀ dialkyl ethers
   mineral oils, such as naphthenic oils, paraffinic oils (petroleum jelly), polybutenes
   organic waxes comprising alkyl chains containing from 4 to 40 carbon atoms,
organosilicon or organic hydrophobic active molecules such as perfuming molecules, anti UV agents, bactericides, and the like.

The invert emulsion (Ei) may be obtained in a conventional manner. For example, it may be obtained by dissolving and/or dispersing the matrix (M) in water and then adding the aqueous solution and/or dispersion obtained to the hydrophobic phase (O), with stirring.

The stirring may be advantageously carried out by means of a paddle frame, a planetary type mixer, a mixer possessing a scraping rotor and a paddle revolving in opposite directions (counter-stirring).

The preparation of the invert emulsion is carried out in general at a temperature greater than the melting point of the material used as hydrophobic phase, but less than that for degradation of the components entering into the composition of the invert emulsion. More particularly, this temperature is between 10 and 80° C.

The duration of stirring may be determined without difficulty by persons skilled in the art. It is preferably sufficient to obtain an average size of aqueous droplets of the invert emulsion of less than 10 μm, as mentioned above. The quantities of the various constituents of the invert emulsion (Ei) have already been defined above.

The matrix (M) according to the invention is equally advantageous for stabilizing multiple emulsions (Em) of the water-in-oil-in-water type.

The multiple emulsion (Em) comprises in particular the invert emulsion (Ei) above, as inner emulsion, dispersed in an aqueous or water-miscible outer phase (We) comprising at lest one dispersing and/or stabilizing agent (De), which dispersant and/or stabilizer (De) may wholly or partly consist of the matrix (M).

Said dispersing and/or stabilizing agent (De) has a hydrophilic tendency.

Preferably, said dispersing and/or stabilizing agent (De) is chosen from hydrophilic surfactants and/or hydrophilic polymers and/or hydrophilic amphiphilic polymers and/or the matrix (M).

The term “hydrophilic” is used in its customary sense of “which has affinity for water”; this means that the dispersing and/or stabilizing agent (De) is not capable of forming a two-phase macroscopic solution in distilled water at 25° C.

Preferably, the outer phase (We) is an aqueous phase.

More particularly, the surfactants and/or polymers (De) satisfy the Bancroft rule and are preferably chosen from compounds which meet both of the two conditions below:
when they are mixed with the outer aqueous phase at a concentration of between 0.1 and 10% by weight of said phase and between 20 and 30° C., they exist in the form of a solution wholly or partly in the concentration range indicated,
when they are mixed with the inner hydrophobic phase (O) at a concentration of between 0.1 and 10% by weight of said phase and between 20 and 30° C., they exist in the form of a dispersion wholly or partly in the concentration range indicated.

The total content of surfactant(s) and/or polymer(s) (De) present in the outer phase (We) may be between 0.01 and 50% by weight, preferably between 0.1 and 10% by weight, more particularly between 0.5 and 5% by weight, relative to the invert emulsion (Ei).

The quantity of outer phase (We) of the multiple emulsion (Em) depends on the concentration desired for the multiple emulsion (Em).

The mass ratio inner invert emulsion (Ei)/outer phase (We) comprising the dispersing and/or stabilizing agent (De) may range from 50/50 to 99/1, preferably from 70/30 to 98/2, most particularly from 70/30 to 80/20.

The mass ratio, expressed on a dry basis, of dispersing and/or stabilizing agent (De)/mass of the inner invert emulsion (Ei) may range from 0.01/100 to 50/100, preferably from 0.1/100 to 10/100, most particularly from 0.5/100 to 5/100.

The dispersing and/or stabilizing agent (De) concentration in the outer phase (We) may range from 1 to 50%.

The average size of the inner invert emulsion (Ei) globules dispersed in the outer phase (We) is preferably less than 200 μm; preferably it may range from 1 to 20 μm, more preferably from 5 to 15 μm.

For good production of the emulsion, the average size of the inner invert emulsion (Ei) globules dispersed in the outer phase (We) is at least twice, preferably at least 5 times, most particularly at least 10 times as large as the average size of the droplets of the inner aqueous phase (Wi) dispersed in the hydrophobic phase.

The multiple emulsion (Em) may be obtained using techniques involving a single reactor or two reactors.

A technique in a single reactor may be carried out using the following steps:
(a) the invert emulsion (Ei) is prepared
(b) the outer phase containing the and/or stabilizing agent (De) is prepared
(c) the outer phase is introduced into the invert emulsion (Ei) without stirring
(d) the whole is stirred.

Step (a) for preparing the invert emulsion (Ei) may be carried out as described above.

Step (b) for preparing the outer phase (We) may be carried out by mixing the constituent of the outer phase (We) (preferably water) and the dispersing and/or stabilizing agent (De).

The outer phase (We) may also comprise adjuvants such as preservatives and osmotic pressure regulating additives.

The preparation of the outer phase may be carried out at room temperature. However, it may be advantageous to prepare the outer phase (We) at a temperature in the region of that at which the invert emulsion (Ei) is prepared.

Once the outer phase (We) has been obtained, it is added to the invert emulsion (Ei) during step (c), without stirring.

Next, after having introduced the entire outer phase (We) into the invert emulsion (Ei), the whole is stirred (step (d).

Advantageously, the stirring is carried out by means of moderately shearing mixers, as is the case for example of stirrers equipped with a paddle frame, planetary type mixers, or those possessing a scraping rotor and a paddle revolving in opposite directions (counterstirring).

This stirring operation preferably takes place at a temperature at which the hydrophobic phase (O) is in a liquid form, and more particularly is between 10 and 80° C.

The average size of the inner invert emulsion (Ei) globules advantageously varies between 1 and 100 atm, more particularly between 1 and 20 μm, advantageously between 5 and 15 μm. The average size of the globules, corresponding to the median diameter by volume (d50), which represents the diameter of the globule equal to 50% of the cumulative distribution, is measured with a Horiba apparatus and/or with an optical microscope.

The various constituents of the emulsion (Em) may be used in the quantities mentioned above.

When (We) is an aqueous phase, although the value of the pH of the aqueous phase is not limiting, it may be advantageous to adjust the pH of the outer aqueous phase by adding a base (sodium hydroxide, potassium hydroxide) or an acid (hydrochloric acid).

By way of illustration, the usual pH range for the outer aqueous phase is between 0 and 14, preferably between 2 and 11, more preferably between 5 and 11.

At the end of this stirring step (d), a concentrated multiple emulsion is obtained whose invert emulsion/outer phase (We) weight ratio may range from 50/50 to 99/1, preferably from 70/30 to 98/2, most particularly from 70/30 to 80/20.

A technique in two reactors may be carried out using the following steps:
(a) the outer phase (We) containing the dispersing and/or stabilizing agent (De) is prepared as above
(b) the invert emulsion (Ei) is prepared as above
(c) the invert emulsion (Ei) is introduced little by little into the outer phase (We), with stirring.

Step (c) for preparing the actual multiple emulsion is carried out with stirring; the stirring may be carried out by means of a paddle frame. Typically, the stirring speed is relatively slow, of the order of 400 revolutions/minute.

The multiple emulsion (Em) obtained is similar to that obtained by the so-called single-reactor technique.

Another technique in two reactors, which makes it possible to prepare a similar multiple emulsion (Em), uses the following steps:
(a) the invert emulsion (Ei) is prepared as above; the invert emulsion (Ei) quantity prepared is divided into two parts
(b) the outer phase (We) containing the dispersing and/or stabilizing agent (De) is prepared
(c) the outer phase (We) is introduced into the first part of the invert emulsion (Ei) without stirring
(d) the whole is stirred
(e) the remaining part of the invert emulsion (Ei) is introduced little by little into the multiple emulsion obtained in step (d), with stirring.

When the dispersing and/or stabilizing agent (De) is a matrix (M), the multiple emulsion (Em) may be directly obtained by subjecting a mixture formed of the constituent(s) of the hydrophobic phase (O), of the matrix (M) and of the constituent(s) of the outer phase (We) to a stirring operation under high shearing.

The following examples are given by way of illustration.

Raw Materials

Polysaccharide (PSA)
“CMA”: cellulose monoacetate having a degree of substitution of 0.61 and a weight-average molar mass of 6400 g/mol
“HEC”: hydroxyethylcellulose Dow Cellosize HEC QP 100M-H marketed by Dow Chemical.

Functional Polyorganosiloxane (POS)
“POS1”: polydimethylsiloxane (linear), with a degree of polymerization of 80, and having an acryloxypropyl functional group at each chain end.
“POS2”: polydimethylsiloxane (linear), with a degree of polymerization of 203, and having an acryloxypropyl functional group at each chain end and an acryloxypropyl functional group in the chain.
“POS3”: polydimethylsiloxane (linear), with a degree of polymerization of 24, and having an epoxycyclohexylethyl functional group at each chain end.

General Method of Irradiation

The powdered mixture of polysaccharide (PSA) and functional polyorganosiloxane (POS) is placed, in a flat homogeneous layer of 1 cm, on a tray. The tray covered with a very thin plastic film is placed on a conveyor belt and conveyed under an electron beam obtained from a 4.5 MeV generator and operating with a 15 mA current.

The conveying speed is adjusted in order to obtain the desired irradiation dose.

The dose D in megarads absorbed is given by the formula D=k(I/V)
k is a constant linked to the apparatus, supplied by the manufacturer
V is the conveying speed in meter/minute
I is the intensity, in milliAmpere, of the output current of the electron beam generator.

1 megarad corresponds to 10 kiloGray.

### EXAMPLE 1

Modification of CMA with POS2—CMA/POS2 Weight Ratio of 10/1—Irradiation of 200 kGy—

The following are successively introduced, with mechanical stirring (of the anchor type), into a 500 ml beaker:
250 g of CMA
25 g of POS2

The mixture is maintained in the air, with stirring, for 3 hours.

The powder obtained is then irradiated under an electron beam as mentioned above, the irradiation dose absorbed being 200 kGy.

### EXAMPLE 2

Modification of CMA with POS2—CMA/POS2 Weight Ratio of 10/1—Irradiation of 30 kGy—

The following are successively introduced, with mechanical stirring (of the anchor type), into a 500 ml beaker:
250 g of CMA
25 g of POS2

The mixture is maintained in the air, with stirring, for 3 hours.

The powder obtained is then irradiated under an electron beam as mentioned above, the irradiation dose absorbed being 30 kGy.

### EXAMPLE 3

Modification of CMA with POS2—CMA/POS2 Weight Ratio of 8/2—Irradiation of 10 kGy—

The following are successively introduced, with mechanical stirring (of the anchor type), into a 500 ml beaker:
240 g of CMA
60 g of POS2

The mixture is maintained in the air, with stirring, for 3 hours.

The powder obtained is then irradiated under an electron beam as mentioned above, the irradiation dose absorbed being 10 kGy.

### EXAMPLE 4

Modification of CMA with POS1—CMA/POS1 Weight Ratio of 9/1—Irradiation of 200 kGy—

The following are successively introduced, with mechanical stirring (of the anchor type), into a 500 ml beaker:
270 g of CMA
30 g of POS1

The mixture is maintained in the air, with stirring, for 3 hours.

The powder obtained is then irradiated under an electron beam as mentioned above, the irradiation dose absorbed being 200 kGy.

### EXAMPLE 5

Modification of CMA with POS1—CMA/POS1 Weight Ratio of 9/1—irradiation of 100 kGy—

The following are successively introduced, with mechanical stirring (of the anchor type), into a 500 ml beaker:
270 g of CMA
30 g of POS1

The mixture is maintained in the air, with stirring, for 3 hours.

The powder obtained is then irradiated under an electron beam as mentioned above, the irradiation dose absorbed being 100 kGy.

### EXAMPLE 6

Modification of CMA with POS1—CMA/POS1 Weight Ratio of 9/1—Irradiation of 30 kGy—

The following are successively introduced, with mechanical stirring (of the anchor type), into a 500 ml beaker:
270 g of CMA
30 g of POS1

The mixture is maintained in the air, with stirring, for 3 hours.

The powder obtained is then irradiated under an electron beam as mentioned above, the irradiation dose absorbed being 30 kGy.

### EXAMPLE 7

Modification of CMA with POS1—CMA/POS1 Weight Ratio of 9/1—Irradiation of 10 kGy—

The following are successively introduced, with mechanical stirring (of the anchor type), into a 500 ml beaker:
270 g of CMA
30 g of POS1

The mixture is maintained in the air, with stirring, for 3 hours.

The powder obtained is then irradiated under an electron beam as mentioned above, the irradiation dose absorbed being 10 kGy.

### EXAMPLE 8

Modification of CMA with POS1—CMA/POS1 Weight Ratio of 8/2—Irradiation of 10 kGy—

The following are successively introduced into a round-bottomed flask for a 500 ml rotary evaporator
170 ml of isopropyl alcohol (98%)
50.71 g of cellulose monoacetate CMA
30 ml of demineralized water
12.5 g of functional silicone oil POS1

The round-bottomed flask is placed on the evaporator and set in rotation (without introducing the vacuum) for one hour at room temperature.

Still in rotation, it is then heated at 45° C. under vacuum, until a dry product is obtained. This operation lasts for about 1 hour.

The drying is completed in a vacuum oven at room temperature. This operation lasts for about 12 hours.

The powder obtained is then irradiated under an electron beam as mentioned above, the irradiation dose absorbed being 10 kGy.

### EXAMPLE 9

Modification of CMA with POS3—CMA/POS2 Weight Ratio of 8/2—Irradiation of 10 kGy—

The following are successively introduced, with mechanical stirring (of the anchor type), into a 500 ml beaker:
240 g of CMA
60 g of POS3

The mixture is maintained in the air, with stirring, for 3 hours.

The powder obtained is then irradiated under an electron beam as mentioned above, the irradiation dose absorbed being 10 kGy.

### EXAMPLE 10

Modification of HEC with POS2—HEC/POS2 Weight Ratio of 85/15—Irradiation of 10 kGy—

The following are successively introduced, with mechanical stirring (of the anchor type), into a 500 ml beaker:
255 g of HEC
45 g of POS2

The mixture is maintained in the air, with stirring, for 3 hours.

The powder obtained is then irradiated under an electron beam as mentioned above, the irradiation dose absorbed being 10 kGy.

### EXAMPLE 11

Modification of HEC with POS3—HEC/POS3 Weight Ratio of 85/15—Irradiation of 10 kGy—

The following are successively introduced, with mechanical stirring (of the anchor type), into a 500 ml beaker:
255 g of HEC
45 g of POS3

The mixture is maintained in the air, with stirring, for 3 hours.

The powder obtained is then irradiated under an electron beam as mentioned above, the irradiation dose absorbed being 10 kGy.

### EXAMPLE 12

Solubility in Water of the Matrices Obtained in Examples 1-11

100 parts by weight of distilled water are introduced into a 1 liter reactor provided with stirring of the paddle frame type (diameter 54 mm, speed 400 revolutions/minute), at room temperature.

10 parts by weight of matrix powder prepared in one of examples 1 to 11 are gradually introduced, with stirring, at room temperature.

The water-solubility of the matrices of examples 1 to 11 is given in the table which follows.

The expression “total solubility” corresponds to a complete dissolution of the matrix, that is to say to the formation of a transparent medium, marginally or slightly colored yellow.

The expression “partial solubility” corresponds to the formation of a gel phase or of an opaque dispersion.

It is observed that a radiation dose well below 100 kGy is favorable for obtaining a matrix which is completely soluble in water.

| | | PSA/POS | Radiation dose | |
|---|---|---|---|---|
| PSA | POS | weight ratio | in kGy | Solubility |
| CMA | POS2 | 10/1 | 200 | partial |
| CMA | POS2 | 10/1 | 30 | total |
| CMA | POS2 | 8/2 | 10 | total |
| CMA | POS1 | 9/1 | 200 | partial |
| CMA | POS1 | 9/1 | 100 | partial |
| CMA | POS1 | 9/1 | 30 | total |
| CMA | POS1 | 9/1 | 10 | total |
| CMA | POS1 | 8/2 | 10 | total |
| CMA | POS3 | 8/2 | 10 | total |
| HEC | POS2 | 85/15 | 10 | total |
| HEC | POS3 | 85/15 | 10 | total |

### EXAMPLE 13

### Stabilization of a Water-in-oil Invert Emulsion

Composition of the Invert Emulsion:
50% by weight of inner aqueous phase consisting of:
   water from 98 to 90 parts by weight
   matrix from 2 to 10 parts by weight
50% by weight of aminated silicone oil phase Rhodorsil® Extrasoft oil marketed by Rhodia
   100 parts by weight

Preparation of the Invert Emulsion:

Preparation of the Inner Aqueous Phase

The water is introduced into a 1 1 reactor equipped with stirring of the paddle frame type (diameter 54 mm, speed 400 revolutions/minute) at room temperature.

The matrix powder is then gradually introduced, with stirring, at room temperature.

The stirring lasts for two hours at room temperature so as to disperse the matrix particles in a homogeneous manner.

The pH of the inner aqueous solution/dispersion is 2 to 3.

Preparation of the Invert Emulsion

The Rhodorsil® Extrasoft oil is introduced into a 2 l reactor equipped with stirring of the paddle frame type (diameter 90 mm, speed 400 revolutions/minute).

The internal aqueous phase is then introduced, over 45 minutes, at room temperature.

The stirring is maintained for 15 minutes in order to refine the emulsion.

An invert emulsion is obtained in which the drops of dispersed aqueous phase have a particle size of 0.5 to 1 μm (observation made by optical microscopy on a sample without and with prior dilution in the Extrasoft oil).

The invert emulsion obtained has the following characteristics:

| | | Content of | | | |
|---|---|---|---|---|---|
| PSA/POS | Radiation | matrix | | | “viscosity” |
| weight | dose | (parts by | | Size | of the |
| ratio | in kGy | weight) | pH | (μm) | emulsion |
| CMA/POS2 | 30 | 2 | 3.1 | 0.6-1.6 | fluid |
| 10/1 | | | | | |
| CMA/POS2 | 10 | 10 | 2.8 | 0.5-0.9 | fluid |
| 8/2 | | | | | |
| CMA/POS1 | 30 | 10 | 2.9 | 0.5-0.9 | fluid |
| 9/1 | | | | | |
| CMA/POS1 | 10 | 10 | 2.8 | 0.5-0.9 | fluid |
| 9/1 | | | | | |
| CMA/POS1 | 10 | 10 | 2.9 | 0.5-0.9 | fluid |
| 8/2 | | | | | |
| CMA/POS3 | 10 | 10 | 2.9 | 0.5-0.9 | fluid |
| 8/2 | | | | | |
| HEC/POS2 | 10 | 10 | 2.9 | 0.5-0.9 | gel |
| 85/15 | | | | | |
| HEC/POS3 | 10 | 10 | 2.8 | 0.5-0.9 | gel |
| 85/15 | | | | | |

1. Matrix (M), which is water-soluble or water-dispersible, made of at least one polysaccharide (PSA) modified under an electron beam with at least one organosilicon compound having at least one functional group and/or at least one functional group capable of reacting and/or interacting with said polysaccharide (PSA), which organosilicon compound is chosen from organosilanes (S) and/or polyorganosiloxanes (POS) having at least one functional group and/or at least one functional group capable of reacting and/or interacting with said polysaccharide (PSA).

2. Matrix according to claim 1, wherein said polysaccharide (PSA) is a nonionic or ionic, linear or branched homopolysaccharide or heteropolysaccharide optionally substituted and/or modified with nonionic or (potentially) ionic groups other than lipophilic polyorganosiloxane groups.

3. Matrix according to claim 1, wherein said polysaccharide (PSA) or its backbone comprises similar or different glycosyl units joined by β(1 -4) bonds, optionally comprising, apart from the β(1-4) bonds, other bonds, preferably β(1-3) and/or β(1 -6).

4. Matrix according to claim 1, wherein said polysaccharide (PSA) has a weight-average molecular mass of 1000 to 5 000 000, preferably of 1000 to 3 000 000 g/mol.

5. Matrix according to claim 3, wherein said similar or different glycosyl units are hexose and/or pentose units.

6. Matrix according to claim 3, wherein said polysaccharide (PSA) or its backbone contains only β(1-4) bonds.

7. Matrix according to claim 6, wherein said polysaccharide (PSA) is a cellulose optionally modified or substituted with one or more nonionic groups, preferably acetate, hydroxyalkyl, hydroxypolyethoxy, (potentially) anionic groups, preferably carboxyalkyl, (potentially) cationic groups, preferably 2-hydroxypropyltrimethylammonium chloride.

8. Matrix according to claim 7, wherein said polysaccharide (PSA) is
a cellulose monoacetate having a degree of substitution of 0.3 to less than 1.2, preferably of 0.3 to 1
a hydroxypropylated cellulose having a degree of modification of 0.2 to 1.5
a hydroxyethylcellulose
a carboxymethylcellulose having a degree of substitution of 0.05 to 1.2, preferably of 0.05 to 1
a 2-hydroxypropyltrimethylammonium chloride cellulose.

9. Matrix according to claim 3, wherein said polysaccharide (PSA) is a galactomannan, preferably a guar gum, optionally modified or substituted with one or more nonionic groups, preferably hydroxyalkyl, (potentially) anionic groups, preferably carboxyalkyl, cationic groups, preferably cationic and/or optionally depolymerized, hydroxyalkylgalactomannan.

10. Matrix according to claim 9, wherein said polysaccharide (PSA) is
a guar gum, preferably as a powder or as split grains
a modified guar, preferably a carboxymethyl or carboxypropyl guar, a carboxymethylhydroxypropyl guar, a hydroxyethyl, hydroxypropyl or hydroxybutyl guar, a hydroxypropyltrimethylammonium chloride guar, most preferably a hydroxypropyl guar having a degree of substitution of less than 0.6
a guar depolymerized by the oxidative route
a hydroxypropylated depolymerized guar having a degree of modification of 0.01 to 0.8
a carboxymethylated depolymerized guar having a degree of substitution of 0.05 to 1.6
a cationized depolymerized guar having a degree of substitution of 0.04 to 0.17, preferably of 0.06 and 0.14.

11. Matrix according to claim 3, wherein said polysaccharide (PSA) is a dextrin optionally containing hydroxyethyl or hydroxypropyl groups or quaternized aminoalkyl groups.

12. Matrix according to claim 1, wherein the organosilicon compound is an organosilane (S) containing from 1 to 3 functional groups capable of reacting or interacting with the polysaccharide (PSA).

13. Matrix according to claim 12, wherein said organosilane (S) has the formula
R¹R′¹R″¹SiY′
the symbol R¹ representing:
a linear or branched alkyl or alkoxy radical containing 1 to 8 carbon atoms, optionally substituted with at least one halogen, preferably fluorine, the alkyl radicals being preferably methyl, ethyl, propyl, octyl, 3,3,3-trifluoropropyl, methoxy, ethoxy, isopropoxy,
an optionally substituted cycloalkyl radical containing between 5 and 8 cyclic carbon atoms,
an aryl radical containing between 6 and 12 carbon atoms which may be substituted, preferably phenyl, tolyl or dichlorophenyl,
an aralkyl part having an alkyl part containing between 5 and 14 carbon atoms and an aryl part containing between 6 and 12 carbon atoms, which is optionally substituted on the aryl part with halogens, alkyls and/or alkoxyls containing 1 to 3 carbon atoms,
the symbols R′¹ and R″¹, which are similar or different, representing R¹ or Y′
the symbol Y′ representing a functional group capable of reacting and/or interacting with the polysaccharide (PSA).

14. Matrix according to claim 13, wherein said organosilane (S) has the formula (CH₃)₃SiY′.

15. Matrix according to claim 1, wherein the organosilicon compound is a functional polyorganosiloxane (POS) which is at least partially linear or cyclic, having at the chain end(s) and/or in the chain one or more functional groups capable of reacting with said polysaccharide (PSA).

16. Matrix according to claim 15, wherein said functional polyorganosiloxane (POS) contains on average from 2 to 1000 siloxy motifs, preferably from 3 to 100 siloxy motifs per macromolecular chain.

17. Matrix according to claim 15, wherein said functional polyorganosiloxane (POS) has on average from 1 to 10, preferably from 1 to 3, more particularly 1 or 2 functional groups capable of reacting with said polysaccharide (PSA).

18. Matrix according to claim 15, wherein said functional polyorganosiloxane (POS) comprises motifs of formula (IV) and/or is terminated by motifs of formula (V) below: 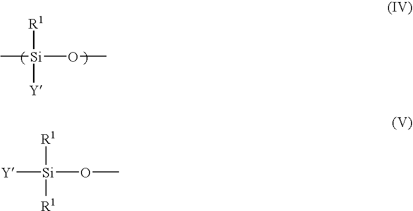
in which formulae:
the symbols R¹ are similar or different and represent:
a linear or branched alkyl or alkoxy radical containing 1 to 8 carbon atoms, optionally substituted with at least one halogen, preferably fluorine, the alkyl radicals being preferably methyl, ethyl, propyl, octyl, 3,3,3-trifluoropropyl, methoxy, ethoxy, isopropoxy,
a cycloalkyl radical containing between 5 and 8 cyclic carbon atoms, which is optionally substituted,
an aryl radical containing between 6 and 12 carbon atoms, which may be substituted, preferably phenyl, tolyl or dichlorophenyl,
an aralkyl part having an alkyl part containing between 5 and 14 carbon atoms and an aryl part containing between 6 and 12 carbon atoms, which is optionally substituted on the aryl part with halogens, alkyls and/or alkoxyls containing 1 to 3 carbon atoms,
the symbols Y′ are similar or different and represent
a radical R¹
a functional group capable of reacting and/or interacting with the polysaccharide (PSA),
at least one of the symbols Y′ being different from R¹.

19. Matrix according to claim 1, wherein said functional group is capable of reacting and/or interacting with said polysaccharide (PSA) according to an ionic or free-radical mechanism.

20. Matrix according to claim 19, wherein said functional group is chosen from epoxy functional groups, vinyl functional groups and alkenyl functional groups, most preferably from epoxy functional groups.

21. Matrix according to claim 20, wherein said functional group is chosen from
the vinyl radical —CH═CH₂,
and/or an epoxy and/or alkenyl and/or alkenyloxy and/or alkenylcarbonyloxy and/or alkenylcarbonylamino radical linked to the silicon atom of the organosilane or to a silicon atom of the polyorganosiloxane via a divalent radical containing from 2 to 20 carbon atoms and which may contain at least one heteroatom, preferably oxygen.

22. Matrix according to claim 21, wherein the epoxy functional group is chosen from those of the following formula:

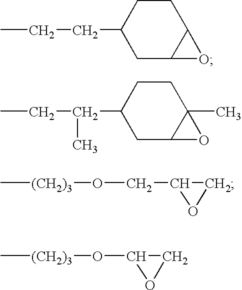

23. Matrix according to claim 21, wherein the alkenyl functional group is chosen from those of the following formula:
—(CH₂)₃—O—CH═CH₂ —(CH₂)₃—O—R²—O—CH═CH₂
—(CH₂)₃—O—CH═CH—R —(CH₂)₃—(OR′²)ₙ—O—CH═CH₂
—(CH₂)₃—O—(O)C—CH ═CH₂ —(CH₂)₃—O—(O)C—C(R)═CH₂
—(CH₂)₃—NH—(O)C—C(R)═CH₂ —(CH₂)₃—NH—(O)C—C(R)═CH₂
in which:
R² represents:
a linear or branched C₁-C₁₂ alkylene radical, which is optionally substituted
or a C₅-C₁₂ arylene radical, preferably phenylene, which is optionally substituted, preferably with one to three C₁-C₆ alkyl groups,
R′² represents a linear or branched C₂-C₃ alkyl radical, with n ranging from 2 to 100,
R represents a linear or branched C₁-C₆ alkyl radical, preferably methyl.

24. Matrix according to claim 1, said matrix being obtained by irradiation, under an electron beam, of a layer, of uniform thickness, of a homogeneous mixture of the polysaccharide (PSA) and of the organosilicon compound having at least one functional group capable of reacting and/or interacting with said polysaccharide (PSA),
the polysaccharide (PSA)/organosilicon compound mass ratio ranging from 1/99 to 99/1, preferably from 70/30 to 90/10
the uniform thickness of the mixture layer ranging up to 3 cm, preferably up to 1.5 cm, more particularly ranging from 100 μm to 1.5 cm
the radiation dose absorbed ranging from 1 to less than 100 kilogray (kGy), preferably from 1 to 50 kGy.

25. Matrix according to claim 24, wherein the organosilicon compound is an organosilane (S), and the polysaccharide (PSA)/organosilane (S) mass ratio is from 50/50 to 99/1, preferably from 70/30 to 90/10.

26. Matrix according to claim 25, wherein the homogeneous mixture exists in solid or liquid form, preferably in solid form, most particularly in the form of a powder.

27. Matrix according to claim 24, wherein the operation for irradiating the mixture itself lasts for less than one second, preferably from 0.001 to 0.5 second.

28. Matrix according to claim 24, wherein the irradiation operation is carried out in the presence of an activator capable of being activated by an electron beam.

29. (canceled)

30. The method according to claim 32, wherein said simple emulsion is a water-in-oil invert emulsion.

31. The method according to claim 32, wherein said multiple emulsion is a water-in-oil-in-water double emulsion.

32. A method for stabilizing a simple or multiple emulsion during the preparation thereof, said method comprising dissolving and/or dispersing the matrix according to claim 1 in water or other aqueous medium and then combining the aqueous solution or dispersion with the other phase or phases.
